(19) United States
(12) Patent Application Publication
(10) Pub. No.: US 2013/0150311 A1
(43) Pub. Date: Jun. 13, 2013

(54) MIXED POLOXAMER EXCIPIENTS

(71) Applicants: Grzegorz Pietrzynski, Montreal (CA); Valery Alakhov, Ile Bizard (CA); Kishore Patel, Pierrfonds (CA)

(72) Inventors: Grzegorz Pietrzynski, Montreal (CA); Valery Alakhov, Ile Bizard (CA); Kishore Patel, Pierrfonds (CA)

(21) Appl. No.: 13/324,395

(22) Filed: Dec. 13, 2011

(51) Int. Cl. A61K 47/34; A61K 31/4545; A61K 31/445; A61K 31/58; A61K 31/495; A61K 31/195; A61K 31/167; A61K 31/4468; A61K 31/53; A61K 31/407; A61K 31/546; A61K 31/7048; A61K 31/496; A61K 31/5377; A61K 31/137; A61K 31/47; A61K 31/64; A61K 31/4525; A61K 31/4439; A61K 31/381; A61K 31/397; A61K 31/4015; A61K 31/517; A61K 31/704; A61K 31/4745; A61K 31/337; A61K 31/7068; A61K 38/13; A61K 31/551; A61P 25/28; A61P 25/04; A61P 37/08; A61P 11/06; A61P 31/04; A61P 3/06; A61P 3/10; A61P 1/08; A61P 9/10; A61P 3/00; A61P 35/00; A61P 25/18; A61P 25/00; A61P 25/26; A61P 37/06; A61P 1/00; A61K 31/56

(52) U.S. Cl. 514/20.5; 514/772; 514/180; 514/290; 514/317; 514/172; 514/255.04; 514/561; 514/626; 514/329; 514/242; 514/210.13; 514/207; 514/29; 514/253.08; 514/236.8; 514/653; 514/311; 514/424; 514/321; 514/342; 514/438; 514/210.02; 514/252.17; 514/34; 514/283; 514/449; 514/49; 514/220; 514/266.4

(57) ABSTRACT

In one aspect, the present invention is directed to a dry, flowable and compressible composition, useful as a pharmaceutical excipient, which composition comprises a mixture of hydrophobic and hydrophilic poly(ethylene oxide)-polypropylene oxide)-poly(ethylene oxide) block copolymers, and which possesses a Carr index of less than about 20. In other aspects, the present invention is directed a pharmaceutical composition comprising such adjuvant and a pharmaceutically active ingredient; as well as to a method of making the compositions.

### FIELD OF THE INVENTION

In one aspect, the present invention is directed to a dry, flowable and compressible composition, useful as a pharmaceutical excipient, which composition comprises a mixture of hydrophobic and hydrophilic poly(ethylene oxide)-poly(propylene oxide)-poly(ethylene oxide) block copolymers, and which possesses a Carr index of less than about 20. In other aspects, the present invention is directed to a pharmaceutical composition comprising the adjuvant and a pharmaceutically active ingredient; as well as to a method of making the compositions.

### BACKGROUND OF THE INVENTION

The use of certain poly(ethylene oxide) -polypropylene oxide) -poly(ethylene oxide) block copolymers having a hydrophobe content of 50% or more by weight as pharmaceutical excipients to enhance the uptake of active materials in certain cell types has been described in a number of publications. See, for example, U.S. Pat. No. 5,840,319; U.S. Pat. No. 6,060,518; Alakhov et al., “Hypersensitization of Multidrug Resistant Human Ovarian Carcinoma Cells by Pluronic P85 Block Coploymer”, Bioconjugate Chem. 7, 209-216 (1996); and Batrakova et al., “Anthracycline antibiotics non-covalently incorporated into copolymer micelles: in vivo evaluation of anti-cancer activity”; British Journal of Cancer 74: 1545-1552 (1996). It is believed that this effect is achieved by the inhibition of ABC mediated efflux mechanisms in such cells. See U.S. Pat. No. 6,387,406.

Unfortunately, such hydrophobic block copolymers (also know an poloxamers; sold under the trade name Pluronics) have been found to aggregate in aqueous solutions at physiological temperatures (see U.S. Pat. No. 6,387,406, Example 34). Such aggregation under physiological conditions can be eliminated by blending such hydrophobic poloxamers with certain hydrophilic poloxamers as described in U.S. Pat. No. 6,387,406.

The use of such mixtures of hydrophilic and hydrophobic copolymers has been shown to greatly increase the effectiveness of certain drugs in clinical studies. Thus, Valle et al.; A phase 2 study of SP1049C, doxorubicin in P-gylcoprotein-targeting pluronics, in patients with advanced adenocarcinoma of the esophagus and gastroesophageal junction; Invest New Drugs; DOI 10.1007/s10637-010-9399-1; published 24 Feb. 2010, describes a Phase II study in which SP 1049C (a composition comprising doxorubicin, hydrophobic Pluronic L61 and hydrophilic Pluronic F127) displayed a response rate of 47% in the evaluable patient population (43% in the ITT formulation). In contrast, Ezdinli et al. “Chemotherapy of Advanced Esophageal Carcinoma”; Cancer 46:2149-2153; 1980; indicates that Adriamycin (a free doxorubicin formulation) elicited a response rate of only 5% when evaluated as a monotherapy in a Phase II study on patients with advanced esophageal cancer (see first full paragraph on page 2152, first column).

Unfortunately, the formulation of hydrophobic and hydrophilic block copolymers employed in the Valle study prepared by mixing Pluronic L61 with Pluronic F127 in an aqueous solution (along with the active material), and freeze drying the mixture to form a waxy pellet does not rapidly dissolve in aqueous solutions. See U.S. Patent Application Publication No. 2007/0196493. Accordingly, such composition requires caution if used in a typical hospital situation, as time must be taken to ensure that the waxy polymeric mixture has fully dissolved in the liquid application medium (typically saline) before administration to patients. U.S. Patent Application Publication No. 2007/0196493 discloses that such waxy mixture will rapidly dissolve in aqueous media if a sugar or similar material, preferably lactose, is incorporated into the polymer matrix by including such material in the aqueous solution which is dried to form the polymeric composition.

While U.S. Patent Application Publication No. 2007/0196493 discloses a mixture which will rapidly dissolve in aqueous media, the waxy nature of such composition makes it unsuitable for use in pills or other similar forms of administration.

Therefore, there is a need for pharmaceutical formulations which can take advantage of the ABC mediated efflux inhibition exhibited by hydrophobic poloxamers (which copolymers are liquids at room temperature), which formulations do not aggregate under physiological conditions, and which are suitable for the production of tablets and other dry forms of application.

Accordingly, it is an object of this invention to provide an excipient comprising such hydrophobic poloxamers, which excipient is in the form of a free flowing, compressible powder.

It is a further object of this invention to provide a method of making the excipient.

### SUMMARY OF THE INVENTION

In one aspect, the present invention is directed to a dry, flowable and compressible composition comprising:

a. a hydrophobic poly(ethylene oxide)-poly(propylene oxide)-poly(ethylene oxide) block copolymer; and

b. a hydrophilic poly(ethylene oxide)-poly(propylene oxide)-poly(ethylene oxide) block copolymer;

characterized in that such composition possesses a Carr index of less than about 20.

In another aspect, the present invention is directed to a pharmaceutical composition comprising the adjuvant and a pharmaceutically active ingredient.

In yet another aspect, this invention is directed to a process for making a dry, flowable and compressible composition comprising the steps of:

a) mixing (i) a hydrophobic poly(ethylene oxide)-polypropylene oxide)-poly(ethylene oxide) block copolymer; and (ii) a hydrophilic poly(ethylene oxide)-polypropylene oxide)-poly(ethylene oxide) block copolymer in an organic solvent to form an organic composition; and

b) drying the organic composition.

### DETAILED DESCRIPTION OF THE INVENTION

In one aspect, the present invention is directed to a dry, flowable and compressible composition comprising:

a. a hydrophobic poly(ethylene oxide)-polypropylene oxide)-poly(ethylene oxide) block copolymer; and

b. a hydrophilic poly(ethylene oxide)-polypropylene oxide)-poly(ethylene oxide) block copolymer;

wherein the composition possesses a Carr index of less than about 20.

Preferably, the composition comprises a mixture selected from the group consisting of:

i. (a) a hydrophobic poly(ethylene oxide)-poly(propylene oxide)-poly(ethylene oxide) block copolymer having an average molecular weight of about 2000 and a hydrophobe weight percentage of about 90% and (b) a hydrophilic poly(ethylene oxide)-poly(propylene oxide)-poly(ethylene oxide) block copolymer having an average molecular weight of about 12,600 and a hydrophobe weight percentage of about 30%, having a w/w proportion of a:b of about 1:4 or higher;

ii. (a) a hydrophobic poly(ethylene oxide)-poly(propylene oxide)-poly(ethylene oxide) block copolymer having an average molecular weight of about 2750 and a hydrophobe weight percentage of about 90% and (b) a hydrophilic poly(ethylene oxide)-poly(propylene oxide)-poly(ethylene oxide) block copolymer having an average molecular weight of about 12,600 and a hydrophobe weight percentage of about 30%, having a w/w proportion of a:b of about 1:3 or higher;

iii. (a) a hydrophobic poly(ethylene oxide)-poly(propylene oxide)-poly(ethylene oxide) block copolymer having an average molecular weight of about 3650 and a hydrophobe weight percentage of about 80% and (b) a hydrophilic poly(ethylene oxide)-poly(propylene oxide)-poly(ethylene oxide) block copolymer having an average molecular weight of about 7,700 and a hydrophobe weight percentage of about 30%, having a w/w proportion of a:b of about 1:4 or higher;

iv. (a) a hydrophobic poly(ethylene oxide)-poly(propylene oxide)-poly(ethylene oxide) block copolymer having an average molecular weight of about 3650 and a hydrophobe weight percentage of about 80% and (b) a hydrophilic poly(ethylene oxide)-poly(propylene oxide)-poly(ethylene oxide) block copolymer having an average molecular weight of about 14,600 and a hydrophobe weight percentage of about 20%, having a w/w proportion of a:b of about 1:3 or higher; and

v. (a) a hydrophobic poly(ethylene oxide)-poly(propylene oxide)-poly(ethylene oxide) block copolymer having an average molecular weight of about 3650 and a hydrophobe weight percentage of about 80% and (b) a hydrophilic poly(ethylene oxide)-poly(propylene oxide)-poly(ethylene oxide) block copolymer having an average molecular weight of about 12,600 and a hydrophobe weight percentage of about 30%, having a w/w proportion of a:b of about 1:4 or higher.

As is employed herein, the phrase “or higher”, when employed in conjunction with a block copolymer ratio, is intended to mean that the second number in the ratio may be higher than the first number presented. Thus, for example, the phrase “1:3 or higher” is intended to include 1:4, but is not intended to include 1:2.

In addition, as is employed herein, the term “about” when employed in conjunction with a value such as a molecular weight or weight percent composition is intended to mean the stated value and a range of values one having ordinary skill in the art would recognize as providing a composition having the properties of the present invention.

Further, as is employed herein, the term “hydrophobe weight percentage” is intended to mean the weight percentage of poly(propylene oxide) contained in the block copolymer. Thus, a “hydrophobic” block copolymer will contain a higher weight percentage of poly(propylene oxide) than poly(ethylene oxide); and a “hydrophilic” block copolymer will contain a higher weight percentage of poly(ethylene oxide) than poly(propylene oxide).

The poly(ethylene oxide)-poly(propylene oxide)-poly(ethylene oxide) block copolymers employed in the composition of the present invention are commercially available under the trademark Pluronic from BASF Corporation. Specifically, the following descriptions above apply to the following Pluronics:

Pluronic L61 A poly(ethylene oxide)-poly(propylene oxide)-poly(ethylene oxide) block copolymer having an average molecular weight of 2000 and a hydrophobe weight percentage of 90%.

Pluronic L81 A poly(ethylene oxide)-poly(propylene oxide)-poly(ethylene oxide) block copolymer having an average molecular weight of 2750 and a hydrophobe weight percentage of 90%.

Pluronic L92 A poly(ethylene oxide)-poly(propylene oxide)-poly(ethylene oxide) block copolymer having an average molecular weight of 3650 and a hydrophobe weight percentage of 80%.

Pluronic F87 A poly(ethylene oxide)-poly(propylene oxide)-poly(ethylene oxide) block copolymer having an average molecular weight of 7700 and a hydrophobe weight percentage of 30%.

Pluronic F108 A poly(ethylene oxide)-poly(propylene oxide)-poly(ethylene oxide) block copolymer having an average molecular weight of 14,600 and a hydrophobe weight percentage of 20%.

Pluronic F127 A poly(ethylene oxide)-poly(propylene oxide)-poly(ethylene oxide) block copolymer having an average molecular weight of 12,600 and a hydrophobe weight percentage of 30%.

Thus, the dry, flowable and compressible excipient compositions of the present invention can comprise the following mixtures:

i. Pluronic L61 with Pluronic F127, w/w proportion of about 1:4 or higher;

ii Pluronic L81 with Pluronic F127, w/w proportion of about 1:3 or higher;

iii. Pluronic L92 with Pluronic F87, w/w proportion of about 1:4 or higher;

iv. Pluronic L92 with Pluronic F108, w/w proportion of about 1:3 or higher; or

v. Pluronic L92 with Pluronic F127, w/w proportion of about 1:4 or higher.

In another aspect, the present invention is directed to a dry, flowable and compressible pharmaceutical composition comprising:

a. a hydrophobic poly(ethylene oxide)-poly(propylene oxide)-poly(ethylene oxide) block copolymer;

b. a hydrophilic poly(ethylene oxide)-poly(propylene oxide)-poly(ethylene oxide) block copolymer; and

c. a pharmaceutically active ingredient;

wherein the composition possesses a Carr index of less than about 20.

Preferably, the pharmaceutical compositions comprise:

(a) an excipient selected from the group consisting of:

i. (a) a hydrophobic poly(ethylene oxide)-poly(propylene oxide)-poly(ethylene oxide) block copolymer having an average molecular weight of about 2000 and a hydrophobe weight percentage of about 90% and (b) a hydrophilic poly(ethylene oxide)-poly(propylene oxide)-poly(ethylene oxide) block copolymer having an average molecular weight of about 12,600 and a hydrophobe weight percentage of about 30%, having a w/w proportion of a:b of about 1:4 or higher;

ii. (a) a hydrophobic poly(ethylene oxide)-poly(propylene oxide)-poly(ethylene oxide) block copolymer having an average molecular weight of about 2750 and a hydrophobe weight percentage of about 90% and (b) a hydrophilic poly(ethylene oxide)-poly(propylene oxide)-poly(ethylene oxide) block copolymer having an average molecular weight of about 12,600 and a hydrophobe weight percentage of about 30%, having a w/w proportion of a:b of about 1:3 or higher;

iii. (a) a hydrophobic poly(ethylene oxide)-poly(propylene oxide)-poly(ethylene oxide) block copolymer having an average molecular weight of about 3650 and a hydrophobe weight percentage of about 80% and (b) a hydrophilic poly(ethylene oxide)-poly(propylene oxide)-poly(ethylene oxide) block copolymer having an average molecular weight of about 7,700 and a hydrophobe weight percentage of about 30%, having a w/w proportion of a:b of about 1:4 or higher;

iv. (a) a hydrophobic poly(ethylene oxide)-poly(propylene oxide)-poly(ethylene oxide) block copolymer having an average molecular weight of about 3650 and a hydrophobe weight percentage of about 80% and (b) a hydrophilic poly(ethylene oxide)-poly(propylene oxide)-poly(ethylene oxide) block copolymer having an average molecular weight of about 14,600 and a hydrophobe weight percentage of about 20%, having a w/w proportion of a:b of about 1:3 or higher; and

v. (a) a hydrophobic poly(ethylene oxide)-poly(propylene oxide)-poly(ethylene oxide) block copolymer having an average molecular weight of about 3650 and a hydrophobe weight percentage of about 80% and (b) a hydrophilic poly(ethylene oxide)-poly(propylene oxide)-poly(ethylene oxide) block copolymer having an average molecular weight of about 12,600 and a hydrophobe weight percentage of about 30%, having a w/w proportion of a:b of about 1:4 or higher; and

(b) a pharmaceutically active ingredient.

The pharmaceutical compositions can comprise the following mixtures:

i. Pluronic L61 with Pluronic F127, w/w proportion of about 1:4 or higher;

ii Pluronic L81 with Pluronic F127, w/w proportion of about 1:3 or higher;

iii. Pluronic L92 with Pluronic F87, w/w proportion of about 1:4 or higher;

iv. Pluronic L92 with Pluronic F108, w/w proportion of about 1:3 or higher; or

v. Pluronic L92 with Pluronic F127, w/w proportion of about 1:4 or higher;

in addition to the pharmaceutically active ingredient.

The composition can have a broad range of hydrophilic poloxamer to hydrophobic poloxamer ratios. In an embodiment, the composition comprises a ratio of hydrophilic poloxamer to hydrophobic poloxamer of from 1:3 to 1:25, including all integer values between 1:3 and 1:25 (e.g., 1:3, 1:4, 1:5, 1:6 and so on up to 1:25). A suitable excipient will comprise at least about 5% by weight of hydrophilic poloxamer (e.g., F127) based upon the total weight of hydrophobic plus hydrophilic poloxamer (a 1:19 w/w proportion); and generally will comprise at least about 10% by weight hydrophilic poloxamer (a 1:9 w/w proportion).

The compositions of the present invention are dry flowing compressible powders possessing Carr Indices of less than about 20, preferably of less than about 10, and most preferably of about 5 or less. Such flowability is unexpected, given that identical mixtures produced employing water rather than an organic solvent exhibit much higher Carr Indices. Consequently, such compositions exhibit unexpectedly desirable compressibility.

Further, it is unexpected that such compositions are free flowing, given that the hydrophobic copolymers employed are liquids while the hydrophilic copolymers employed are waxy solids.

In general, any pharmaceutically active ingredient may be employed. Typically, such active ingredient can comprise any active molecule which has limited solubility in aqueous solutions and/or which becomes destabilized in a reactive environment. The active ingredient can be in the form of a pharmaceutically acceptable salt.

Suitable active ingredients which can be employed in the practice of this invention are: Alzheimer treatments such as donepezil; analgesics such as lamotrigine, fentanyl, lidocaine, and gabapentin; antiallergics such as cetirizine, mometasone, fexofenadine, desloratadine, fluticasone and loratadine; antiasthmatics such as montelukast, budesonide, fluticasone, and levalbuterol; antibacterials such as clarithromycin, linezolid, ciprofloxacin, azithromycin, cefdinir, and meropenem; anticholesteremic drugs such as atorvastatin, simvastatin, rosuvastatin, ezetimibe, fenofibrate, pravastatin and fluvastatin; antidepressants such as escitalopram, sertraline, duloxetine, and paroxetine; antidiabetics such as rosiglitazone and glimepiride; antiemetics such as ondansetron, terbinafine, voriconazole, and fluconazole; antihypertensives such as amlodipine, valsartan, losartan, irbesartan, metoprolol, candesartan, telmisartan, latanoprost, carvedilol, olmesartan, ramipril, nifedipine, bosentan, ramipril, enalapril, doxazosin, aand bisoprolol; antihypocalcemics such asraloxifene; anti-inflammatories such as celecoxib and meloxicam; antineoplastics such as doxorubicin, bendamustine, SN-38, docetaxel, anastrozole, gemcitabine, bicalutamide, tamsulosin, irinotecan, letrozole, temozolomide, erlotinib, finasteride and paclitaxel; antiobesity agents such as orlistat; antiplatelet agents such as clopidogrel; antipsychotics such as olanzapine, risperidone, quetiapine, aripiprazole and ziprasidone; antispasmodics such as tolterodine; antithyroids such as levothyroxine antivirals such as lopinavir, atazanavir and efavirenz; central nervous system stimulants such as methylphenidate, modafinil and eszopiclone; contraceptives such as drospirenone; dietary supplements such as oxcarbazepine; erectile dysfunction treatments such as sildenafil, tadalafil and vardenafil; gastrointestinal agents such as esomeprazole, lansoprazole, rabeprazole, omeprazole, tegaserod and famotidine; and immunosuppresives such as tacrolimus, cyclosporin, rapamycin and thalidomide.

The pharmaceutical compositions will comprise a sufficient amount of active ingredient such that a therapeutical dosage can be provided to a patient, which amounts can be readily determined by one of ordinary skill in the art. It will be understood that the precise dosage will vary with age, size, sex and condition of the subject as well as the severity of the disorder to be treated and the like and be subject to the physician's discretion.

The amount of excipient to pharmaceutical active will depend greatly upon the particular active ingredient employed. Typically, the weight ratio of excipient to active ingredient will range between about 100:1 and about 1:10; more typically such ratio will range between about 75:1 and about 1:1; and will frequently range between about 50:1 and 5:1.

In addition to the poloxamer excipient and active ingredient, the pharmaceutical compositions of this invention may further contain pharmaceutically acceptable excipients, such as sugars, polyalcohols, soluble polymers, salts and lipids. Sugars and polyalcohols which may be employed include, without limitation, lactose, sucrose, mannitol, and sorbitol. Illustrative of the soluble polymers which may be employed are polyoxyethylene, poloxamers, polyvinylpyrrolidone, and dextran. Useful salts include, without limitation, sodium chloride, magnesium chloride, and calcium chloride. Lipids which may be employed include, without limitation, fatty acids esters, glycolipids, and phospholipids.

The dry flowable and compressible excipients of this invention are prepared by the steps of:

a. mixing a (i) a hydrophobic poly(ethylene oxide)-polypropylene oxide)-poly(ethylene oxide) block copolymer; and (ii) a hydrophilic poly(ethylene oxide)-poly(propylene oxide)-poly(ethylene oxide) block copolymer; in an organic solvent to form an organic mixture; and

b. drying the organic mixture.

Preferably, the compositions are formed by:

(A) mixing a combination of poly(ethylene oxide)-polypropylene oxide)-poly(ethylene oxide) block copolymers selected from the group consisting of:

i. (a) a poly(ethylene oxide)-polypropylene oxide)-poly(ethylene oxide) block copolymer having an average molecular weight of about 2000 and a hydrophobe weight percentage of about 90% and (b) a poly(ethylene oxide)-polypropylene oxide)-poly(ethylene oxide) block copolymer having an average molecular weight of about 12,600 and a hydrophobe weight percentage of about 30%, having a w/w proportion of a:b of about 1:4 or higher;

ii. (a) a poly(ethylene oxide)-polypropylene oxide)-poly(ethylene oxide) block copolymer having an average molecular weight of about 2750 and a hydrophobe weight percentage of about 90% and (b) a poly(ethylene oxide)-poly(propylene oxide)-poly(ethylene oxide) block copolymer having an average molecular weight of about 12,600 and a hydrophobe weight percentage of about 30%, having a w/w proportion of a:b of about 1:3 or higher;

iii. (a) a poly(ethylene oxide)-poly(propylene oxide)-poly(ethylene oxide) block copolymer having an average molecular weight of about 3650 and a hydrophobe weight percentage of about 80% and (b) a poly(ethylene oxide)-poly(propylene oxide)-poly(ethylene oxide) block copolymer having an average molecular weight of about 7,700 and a hydrophobe weight percentage of about 30%, having a w/w proportion of a:b of about 1:4 or higher;

iv. (a) a poly(ethylene oxide)-poly(propylene oxide)-poly(ethylene oxide) block copolymer having an average molecular weight of about 3650 and a hydrophobe weight percentage of about 80% and (b) a poly(ethylene oxide)-poly(propylene oxide)-poly(ethylene oxide) block copolymer having an average molecular weight of about 14,600 and a hydrophobe weight percentage of about 20%, having a w/w proportion of a:b of about 1:3 or higher; and

v. (a) a poly(ethylene oxide)-poly(propylene oxide)-poly(ethylene oxide) block copolymer having an average molecular weight of about 3650 and a hydrophobe weight percentage of about 80% and (b) a poly(ethylene oxide)-poly(propylene oxide)-poly(ethylene oxide) block copolymer having an average molecular weight of about 12,600 and a hydrophobe weight percentage of about 30%, having a w/w proportion of a:b of about 1:4 or higher; in an organic solvent to form an organic mixture; and

(B) drying the organic mixture.

Most preferably, the excipient compositions are prepared by:

a. mixing a member selected from the group consisting of mixtures of:

i. Pluronic L61 with Pluronic F127, w/w proportion of about 1:4 or higher;

ii Pluronic L81 with Pluronic F127, w/w proportion of about 1:3 or higher;

iii. Pluronic L92 with Pluronic F87, w/w proportion of about 1:4 or higher;

iv. Pluronic L92 with Pluronic F108, w/w proportion of about 1:3 or higher; and

v. Pluronic L92 with Pluronic F127, w/w proportion of about 1:4 or higher; with an organic solvent to form an organic composition; and

b. drying the organic composition.

Preferred organic solvents which can be employed include alcohols, particularly ethanol, and halogenated hydrocarbons, particularly dichloromethane. The mixing is typically conducted at room temperature and pressure, although higher or lower temperatures and/or pressures can be employed.

In certain embodiments, it can be useful for a minor amount of water to be blended with the organic solvent. Typically, in such situations, the water will comprise less than about 25% by weight, preferably less than about 10% by weight of the water/organic solvent mixture.

Any conventional method of drying can be utilized. The preferred methods of drying are drying in vacuum and spray drying. In utilizing these methods any of the conventional techniques for carrying them out may be employed.

The pharmaceutical compositions of this invention may be prepared by either including the pharmaceutically active ingredient in the organic solvent/block copolymer mixture prior to drying; or be first forming the dry, flowable, compressible excipient, and then blending such excipient with the pharmaceutically active ingredient.

The invention can be further illustrated by the following examples thereof, although it will be understood that these examples are included merely for purposes of illustration and are not intended to limit the scope of the invention unless otherwise specifically indicated. All percentages, ratios, and parts herein, in the Specification, Examples, and Claims, are by weight and are approximations unless otherwise stated.

### EXAMPLES

### Example 1

### Effects of Pluronics as Inhibitors of ABC Efflux Mechanisms

Confluent mono layers of MCF-7 ADR adriamycin resistant cells were washed, trypsinized with typsin-EDTA, and washed with PBS pH 7.2. The cells after washing were distributed into 5 ml polystyrene tubes, 5×10⁵ cells (500 microliter) per tube. The tubes were centrifuged at 1200 RPM and PBS was decanted. The cells were then re-suspended in PBS containing 500 nM Rhodamine 123 and various concentrations of Pluronics, as listed in Table 1 below; reference samples with no Pluronic, and with 0.05% L61 were included in each tested series. All samples were prepared and tested in triplicates, and the presented results represent the average. Cells were incubated at 37° C. with 5% CO₂ for 45 minutes. After the incubation the samples were cooled to 4° C., centrifuged at 1200 RPM, washed once with 4 ml cold PBS, and re-suspended in 500 microliter cold PBS. Cells suspension, 300 microliter /well were transferred into 96-well polystyrene plates and the fluorescence was measured λ_{EX} 485 nm, and emission X_{Em} 530 nm. The increased fluorescence of the samples incubated with Pluronics vs. fluorescence of samples not containing Pluronics is interpreted as the enhancement of Rhodamine 123 uptake. This enhancement is normalized to the effect caused by 0.05% solution of Pluronic L61.

TABLE 1
Effect of Pluronics on uptake of Rhodamine 123 into MCF-7 ADR cells

| | Pluronic concentration [%] | | | | | | | | |
|---|---|---|---|---|---|---|---|---|---|
| Pluronic | 0.0005 | 0.0015 | 0.005 | 0.015 | 0.05 | 0.15 | 0.5 | Equivalent | |
| name | Relative Rhodamine 123 uptake enhancement | | | | | | | Conc.* | Potency** |
| L31 | 0.011 | 0.000 | 0.044 | 0.261 | 0.455 | 0.588 | 0.657 | 0.13% | 4 |
| L61 | 0.014 | 0.055 | 0.483 | 0.854 | 1.000 | 1.053 | | 0.007% | 70 |
| L62 | 0.047 | 0.007 | 0.088 | 0.602 | 1.183 | | | 0.014% | 35 |
| L64 | | 0.021 | 0.032 | 0.167 | 0.646 | 0.907 | | 0.04% | 11 |
| L81 | 0.055 | 0.253 | 0.938 | 1.228 | 1.091 | | | 0.003% | 162 |
| P84 | | 0.008 | 0.001 | 0.026 | 0.316 | 0.925 | | 0.09% | 6 |
| P85 | | 0.009 | 0.035 | 0.065 | 0.215 | 0.469 | 0.564 | 0.5% | 1 |
| L92 | 0.012 | 0.092 | 0.669 | 1.251 | | | | 0.004% | 115 |
| L101 | | 0.028 | 0.206 | 0.999 | 1.095 | | | 0.01% | 53 |
| P103 | | 0.039 | 0.137 | 0.292 | 1.367 | | | 0.024% | 21 |
| P104 | | 0.000 | 0.059 | 0.106 | 0.171 | 0.290 | 0.690 | 0.4% | 1 |
| L121 | | 0.039 | 0.079 | 0.260 | 0.849 | 0.947 | | 0.03% | 15 |
| P123 | | 0.000 | 0.010 | 0.053 | 0.634 | 1.115 | 1.141 | 0.05% | 11 |

*Equivalent concentration—concentration of the polymer of the potency equivalent to 0.5% Pluronic P85, determined from interpolation of experimental data
**Potency in Rhodamine 123 uptake enhancement, relative to 0.5% Pluronic P85

The above results demonstrate that multiple polymers of the structure POE-POP-POE cause enhancement of Rhodamine 123 uptake to MCF-7 ADR cells, interpreted as inhibition of ABC efflux mechanisms in these cells. The particularly effective Pluronics are L81, L92 and L61.

### Example 2

### Adhesiveness of Pluronic Mixtures

Mixtures of the below listed Pluronics were prepared employing either methanol or dichloromethane as a solvent using the following procedure. Precisely weighted amounts of Pluronics, as described in Table 2 below, were placed in graduated plastic vials. 5 mL of either anhydrous ethanol or dichloromethane were added to each vial, and the materials were mixed until homogenous. The mixtures were then placed in a SpeedVac system and the solvent removed under reduced pressure while centrifuging the samples to prevent spill. The residue materials were crushed with a glass rod to convert solids into powder. The material from each vial was subsequently transferred into another weighted container by tapping the capsized vial, and amount transferred was determined by weight. The material was presumed non-adhesive if at least 95% of the mass has been transferred. The results are listed in Table 2 below.

TABLE 2

| | | | | | Pro- | | Dichloro- |
|---|---|---|---|---|---|---|---|
| | Hydro- | | Hydro- | | portion | Ethanol | methane |
| No. | phobic | [g] | philic | [g] | (w/w) | Adhesive | Adhesive |
| 1 | L61 | 0.11 | F127 | 0.89 | 1:8 | No | No |
| 2 | L61 | 0.20 | F127 | 0.80 | 1:4 | No | No |
| A1 | L61 | 0.25 | F127 | 0.75 | 1:3 | Yes | Yes |
| A2 | L61 | 0.33 | F127 | 0.67 | 1:2 | Yes | Yes |
| 3 | L81 | 0.11 | F127 | 0.89 | 1:8 | No | No |
| 4 | L81 | 0.20 | F127 | 0.80 | 1:4 | No | No |
| B1 | L81 | 0.25 | F127 | 0.75 | 1:3 | Yes | Yes |
| B2 | L81 | 0.33 | F127 | 0.67 | 1:2 | Yes | Yes |
| 5 | L92 | 0.10 | F87 | 0.90 | 1:9 | No | No |
| 6 | L92 | 0.11 | F87 | 0.89 | 1:8 | No | No |
| 7 | L92 | 0.20 | F87 | 0.80 | 1:4 | No | No |
| C1 | L92 | 0.25 | F87 | 0.75 | 1:3 | Yes | Yes |
| C2 | L92 | 0.33 | F87 | 0.67 | 1:2 | Yes | Yes |
| 8 | L92 | 0.10 | F108 | 0.90 | 1:9 | No | No |
| 9 | L92 | 0.11 | F108 | 0.89 | 1:8 | No | No |
| 10 | L92 | 0.20 | F108 | 0.80 | 1:4 | No | No |
| D1 | L92 | 0.25 | F108 | 0.75 | 1:3 | No | Yes |
| D2 | L92 | 0.33 | F108 | 0.67 | 1:2 | Yes | Yes |
| 11 | L92 | 0.10 | F127 | 0.90 | 1:9 | No | No |
| 12 | L92 | 0.11 | F127 | 0.89 | 1:8 | No | No |
| 13 | L92 | 0.20 | F127 | 0.80 | 1:4 | No | No |
| 14 | L92 | 0.25 | F127 | 0.75 | 1:3 | No | No |
| E1 | L92 | 0.33 | F127 | 0.67 | 1:2 | Yes | Yes |

The above results demonstrate that the compositions of the present invention are flowable, non-adhesive powders.

### Example 3

### Compressibility of Pluronic Mixtures (as Indicated by Carr Index)

The bulk density (ρ_{B}), tap density (ρ_{T}) of each of the non-adhesive compositions produced above were measured, and the Carr Index C=100*(1−ρ_{B}/ρ_{T}) calculated. As a comparison, identical compositions were prepared employing water as the solvent, followed by freeze drying of the aqueous solution. The Carr Indices of such compositions were similarly calculated. The results of such measurements are provided in Table 3 below.

TABLE 3

| | | | | | | Carr Index | | |
|---|---|---|---|---|---|---|---|---|
| | Hydro- | | Hydro- | | Proportion | | Dichloro- | |
| No. | phobic | [g] | philic | [g] | (w/w) | Ethanol | methane | Water |
| 1 | L61 | 0.11 | F127 | 0.89 | 1:8 | 5.26 | 10.00 | 22.3 |
| 2 | L61 | 0.20 | F127 | 0.80 | 1:4 | 16.67 | 9.09 | 28.0 |
| 3 | L81 | 0.11 | F127 | 0.89 | 1:8 | 0.00 | 5.00 | 16.8 |
| 4 | L81 | 0.20 | F127 | 0.80 | 1:4 | 0.00 | 8.00 | 22.0 |
| 5 | L92 | 0.10 | F87 | 0.90 | 1:9 | 5.00 | 0.00 | 20.0 |
| 6 | L92 | 0.11 | F87 | 0.89 | 1:8 | 5.00 | 0.00 | 31.4 |
| 7 | L92 | 0.20 | F87 | 0.80 | 1:4 | 9.09 | 8.70 | 28.6 |
| 8 | L92 | 0.10 | F108 | 0.90 | 1:9 | 8.70 | 5.00 | 13.0 |
| 9 | L92 | 0.11 | F108 | 0.89 | 1:8 | 8.70 | 10.00 | 13.6 |
| 10 | L92 | 0.20 | F108 | 0.80 | 1:4 | 8.33 | 5.00 | 23.2 |
| 11 | L92 | 0.10 | F127 | 0.90 | 1:9 | 5.26 | 5.00 | 10.0 |
| 12 | L92 | 0.11 | F127 | 0.89 | 1:8 | 5.00 | 5.00 | 16.0 |
| 13 | L92 | 0.20 | F127 | 0.80 | 1:4 | 5.26 | 5.00 | 24.6 |
| 14 | L92 | 0.25 | F127 | 0.75 | 1:3 | 0.00 | 13.04 | 28.9 |

The above results show that the powders produced by the process of this invention exhibit unexpectedly lower Carr Indices, and are thus more compressible.

What is claimed is:

1. A dry, flowable and compressible composition comprising:
a. a hydrophobic poly(ethylene oxide)-polypropylene oxide)-poly(ethylene oxide) block copolymer; and
b. a hydrophilic poly(ethylene oxide)-polypropylene oxide)-poly(ethylene oxide) block copolymer;
wherein the composition possesses a Carr index of less than about 20.

2. The composition of claim 1, wherein said composition comprises a mixture selected from the group consisting of:
i. (a) a hydrophobic poly(ethylene oxide)-polypropylene oxide)-poly(ethylene oxide) block copolymer having an average molecular weight of about 2000 and a hydrophobe weight percentage of about 90% and (b) a hydrophilic poly(ethylene oxide)-polypropylene oxide)-poly(ethylene oxide) block copolymer having an average molecular weight of about 12,600 and a hydrophobe weight percentage of about 30%, having a w/w proportion of a:b of about 1:4 or higher;
ii. (a) a hydrophobic poly(ethylene oxide)-polypropylene oxide)-poly(ethylene oxide) block copolymer having an average molecular weight of about 2750 and a hydrophobe weight percentage of about 90% and (b) a hydrophilic poly(ethylene oxide)-polypropylene oxide)-poly(ethylene oxide) block copolymer having an average molecular weight of about 12,600 and a hydrophobe weight percentage of about 30%, having a w/w proportion of a:b of about 1:3 or higher;
iii. (a) a hydrophobic poly(ethylene oxide)-polypropylene oxide)-poly(ethylene oxide) block copolymer having an average molecular weight of about 3650 and a hydrophobe weight percentage of about 80% and (b) a hydrophilic poly(ethylene oxide)-polypropylene oxide)-poly(ethylene oxide) block copolymer having an average molecular weight of about 7,700 and a hydrophobe weight percentage of about 30%, having a w/w proportion of a:b of or higher about 1:4 or higher;
iv. (a) a hydrophobic poly(ethylene oxide)-polypropylene oxide)-poly(ethylene oxide) block copolymer having an average molecular weight of about 3650 and a hydrophobe weight percentage of about 80% and (b) a hydrophilic poly(ethylene oxide)-poly(propylene oxide)-poly(ethylene oxide) block copolymer having an average molecular weight of about 14,600 and a hydrophobe weight percentage of about 20%, having a w/w proportion of a:b of about 1:3 or higher; and
v. (a) a hydrophobic poly(ethylene oxide)-poly(propylene oxide)-poly(ethylene oxide) block copolymer having an average molecular weight of about 3650 and a hydrophobe weight percentage of about 80% and (b) a hydrophilic poly(ethylene oxide)-poly(propylene oxide)-poly(ethylene oxide) block copolymer having an average molecular weight of about 12,600 and a hydrophobe weight percentage of about 30%, having a w/w proportion of a:b of about 1:4 or higher.

3. The composition of claim 1 wherein such composition comprises a mixture selected from the group consisting of:
i. Pluronic L61 with Pluronic F127 having w/w proportion of about 1:4 or higher;
ii Pluronic L81 with Pluronic F127 having w/w proportion of about 1:3 or higher;
iii. Pluronic L92 with Pluronic F87 having w/w proportion of about 1:4 or higher;
iv. Pluronic L92 with Pluronic F108 having w/w proportion of about 1:3 or higher; and
v. Pluronic L92 with Pluronic F127 having w/w proportion of about 1:4 or higher.

4. The composition of claim 1 wherein said composition comprises at least about 5% by weight of a hydrophilic poly(ethylene oxide)-poly(propylene oxide)-poly(ethylene oxide) block copolymer, based upon the total weight of the hydrophilic poly(ethylene oxide)-poly(propylene oxide)-poly(ethylene oxide) block copolymer plus the hydrophobic poly(ethylene oxide)-poly(propylene oxide)-poly(ethylene oxide) block copolymer.

5. The composition of claim 3 wherein said composition comprises at least about 10% by weight of a hydrophilic poly(ethylene oxide)-poly(propylene oxide)-poly(ethylene oxide) block copolymer, based upon the total weight of the hydrophilic poly(ethylene oxide)-poly(propylene oxide)-poly(ethylene oxide) block copolymer plus the hydrophobic poly(ethylene oxide)-poly(propylene oxide)-poly(ethylene oxide) block copolymer.

6. The composition of claim 1 wherein said composition has a Carr Index of less than about 10.

7. The composition of claim 6 wherein said composition has a Carr Index of less than about 5.

8. A pharmaceutical composition comprising (a) the composition of claim 1; and (b) a pharmaceutically active ingredient.

9. The pharmaceutical composition of claim 8 wherein the pharamaceutically active ingredient is selected from the group consisting of Alzheimer treatments, analgesics, antiallergics, antiasthmatics, antibacterials, anticholesteremic drugs, antidepressants, antidiabetics, antiemetics, antihypertensives, antihypocalcemics, anti-inflammatories, antineoplastics, antiobesity agents, antiplatelet agents, antipsychotics, antispasmodics, antithyroids, antivirals, central nervous system stimulants, contraceptives, dietary supplements, erectile dysfunction treatments, gastrointestinal agents and immunosuppresives.

10. The pharmaceutical composition of claim 8 wherein the pharamaceutically active ingredient is selected from the group consisting of donepezil, lamotrigine, fentanyl, lidocaine, gabapentin, cetirizine, mometasone, fexofenadine, desloratadine, fluticasone, loratadine, montelukast, budesonide, fluticasone, levalbuterol, clarithromycin, linezolid, ciprofloxacin, azithromycin, cefdinir, meropenem, atorvastatin, simvastatin, rosuvastatin, ezetimibe, fenofibrate, pravastatin, fluvastatin, escitalopram, sertraline, duloxetine, paroxetine, rosiglitazone, glimepiride, ondansetron, terbinafine, voriconazole, fluconazole, amlodipine, valsartan, losartan, irbesartan, metoprolol, candesartan, telmisartan, latanoprost, carvedilol, olmesartan, ramipril, nifedipine, bosentan, ramipril, enalapril, doxazosin, bisoprolol, asraloxifene, celecoxib, meloxicam, doxorubicin, bendamustine, SN-38, docetaxel, anastrozole, gemcitabine, bicalutamide, tamsulosin, irinotecan, letrozole, temozolomide, erlotinib, finasteride, paclitaxel, orlistat, clopidogrel, olanzapine, risperidone, quetiapine, aripiprazole, ziprasidone, tolterodine, levothyroxine, lopinavir, atazanavir, efavirenz, methylphenidate, modafinil, eszopiclone, drospirenone, oxcarbazepine, sildenafil, tadalafil,vardenafil, lansoprazole, rabeprazole, omeprazole, tegaserod, famotidine, tacrolimus, cyclosporin and thalidomide.

11. A process for preparing a dry flowable and compressible excipient having a Carr index of less than about 20 comprising the steps of:
a. mixing a (i) a hydrophobic poly(ethylene oxide)-poly(propylene oxide)-poly(ethylene oxide) block copolymer; and (ii) a hydrophilic poly(ethylene oxide)-poly(propylene oxide)-poly(ethylene oxide) block copolymer; in an organic solvent to form an organic mixture; and
b. drying the organic mixture.

12. The process of claim 11 wherein the poly(ethylene oxide)-poly(propylene oxide)-poly(ethylene oxide) block copolymers are selected from the group consisting of:
i. (a) a hydrophobic poly(ethylene oxide)-poly(propylene oxide)-poly(ethylene oxide) block copolymer having an average molecular weight of about 2000 and a hydrophobe weight percentage of about 90% and (b) a hydrophilic poly(ethylene oxide)-poly(propylene oxide)-poly(ethylene oxide) block copolymer having an average molecular weight of about 12,600 and a hydrophobe weight percentage of about 30%, having a w/w proportion of a:b of about 1:4 or higher;
ii. (a) a hydrophobic poly(ethylene oxide)-poly(propylene oxide)-poly(ethylene oxide) block copolymer having an average molecular weight of about 2750 and a hydrophobe weight percentage of about 90% and (b) a hydrophilic poly(ethylene oxide)-poly(propylene oxide)-poly(ethylene oxide) block copolymer having an average molecular weight of about 12,600 and a hydrophobe weight percentage of about 30%, having a w/w proportion of a:b of about 1:3 or higher;
iii. (a) a hydrophobic poly(ethylene oxide)-poly(propylene oxide)-poly(ethylene oxide) block copolymer having an average molecular weight of about 3650 and a hydrophobe weight percentage of about 80% and (b) a hydrophilic poly(ethylene oxide)-poly(propylene oxide)-poly(ethylene oxide) block copolymer having an average molecular weight of about 7,700 and a hydrophobe weight percentage of about 30%, having a w/w proportion of a:b of about 1:4 or higher;
iv. (a) a hydrophobic poly(ethylene oxide)-poly(propylene oxide)-poly(ethylene oxide) block copolymer having an average molecular weight of about 3650 and a hydrophobe weight percentage of about 80% and (b) a hydrophilic poly(ethylene oxide)-polypropylene oxide)-poly(ethylene oxide) block copolymer having an average molecular weight of about 14,600 and a hydrophobe weight percentage of about 20%, having a w/w proportion of a:b of about 1:3 or higher; and
v. (a) a hydrophobic poly(ethylene oxide)-polypropylene oxide)-poly(ethylene oxide) block copolymer having an average molecular weight of about 3650 and a hydrophobe weight percentage of about 80% and (b) a hydrophilic poly(ethylene oxide)-polypropylene oxide)-poly(ethylene oxide) block copolymer having an average molecular weight of about 12,600 and a hydrophobe weight percentage of about 30%, having a w/w proportion of a:b of about 1:4 or higher.

13. The process of claim 11 wherein step (a) comprises the mixing of a mixture selected from the group consisting of:
i. Pluronic L61 with Pluronic F127 having w/w proportion of about 1:4 or higher;
ii Pluronic L81 with Pluronic F127 having w/w proportion of about 1:3 or higher;
iii. Pluronic L92 with Pluronic F87 having w/w proportion of about 1:4 or higher;
iv. Pluronic L92 with Pluronic F108 having w/w proportion of about 1:3 or higher; and
v. Pluronic L92 with Pluronic F127 having w/w proportion of about 1:4 or higher.

14. The process of claim 11 wherein the organic solvent is selected from the group consisting of alcohols and halogenated hydrocarbons.

15. The process of claim 11 wherein the organic solvent is ethanol.

16. The process of claim 11 wherein the organic solvent is dichloromethane.

17. The process of claim 11 wherein a minor amount of water is added to the organic solvent.

18. The process of claim 15 wherein the water comprises less than about 25% by weight of the organic solvent water mixture.

19. The process of claim 15 wherein the water comprises less than about 10% by weight of the organic solvent water mixture.

20. The process of claim 11 where no water is added to the organic solvent.
